(19) United States
(12) Patent Application Publication
(10) Pub. No.: US 2022/0313273 A1
(43) Pub. Date: Oct. 6, 2022

(54) SYSTEM AND METHOD FOR TEMPORARILY STOPPING BLOOD FLOW THROUGH A BLOOD VESSEL

(71) Applicant: Yair GALILI, Or Yehuda (IL)

(72) Inventor: Yair GALILI, Or Yehuda (IL)

(21) Appl. No.: 17/641,456

(22) PCT Filed: Sep. 16, 2020
(86) PCT No.: PCT/IL2020/051020

§ 371 Date: Mar. 9, 2022

Related U.S. Application Data
(60) Provisional application No. 62/901,352, filed on Sep. 17, 2019.

(51) Int. Cl. A61B 17/12; A61B 17/00; A61F 5/00

(52) U.S. Cl. CPC A61B 17/12136; A61B 17/00234; A61F 5/003; A61B 2017/00876

(57) ABSTRACT

The present subject matter provides a system for temporarily stopping blood flow through a blood vessel, the system including: at least one magnetically attractive element configured to be introduced into an internal part of a body of a patient, and temporarily placed near a first blood vessel that supplies blood to at least one second blood vessel; and at least one magnetic field generator configured to be placed outside a body of the patient, and generate a magnetic field that attracts the at least one magnetically attractive element in a manner that the at least one magnetically attractive element presses the first blood vessel, thereby stopping flow of blood through the first blood vessel toward the second blood vessel. Additional embodiments of the system, of a kit comprising components of the same, and a method for temporarily stopping blood flow through a blood vessel, are disclosed herein as well.

### CROSS-REFERENCE TO RELATED APPLICATION

This application claims priority to U.S. Provisional Patent Application No. 62/901,352, filed Sep. 17, 2019, the contents of which is incorporated herein by reference in its entirety.

### FIELD

The present subject matter relates to cardiovascular treatment. More particularly, the present subject matter relates to temporarily stopping blood flow through a blood vessel.

### BACKGROUND

Internal bleeding, for example non-compressible torso hemorrhage, is one of the leading causes of death due to vessel injuries among combat patients. In 2012, a peer-reviewed study by Army surgical researchers, of morm than 5.000 war deaths, found that more than 90% of the preventable fatalities were from exsanguination. (Brian J Eastridge, Robert L Mabry, Peter Seguin, Joyce Cantrell. Terrill Tops, Paul Uribe. Olga Mallett. Tamara Zubko, Lynne Oetjen-Gerdes. Todd E Rasmussen, Frank K Butler. Russ S Kotwal, John B Holcomb. Charles Wade. Howard Champion. Mimi Lawnick. Leon Moores, Lome H Blackbourne, Death on the battlefield (2001-2011): implications for the future of combat casualty care. Trauma Acute Care Surg. 2012 December; 73[6 Suppl 5]: S431-437, the entire contents of which is incorporated herein by reference). Internal bleeding is also one of the leading causes of death due to vessel injuries among civilian trauma patients. Ruptured abdominal aortic aneurysm and post-partum bleeding are examples of non-traumatic causes of non-compressible torso hemorrhage.

Current treatment for non-compressible torso hemorrhage includes rapid transport of the patient to a hospital and emergent intervention. Several early hemorrhage control interventions which can improve survival are: placement of a resuscitative endovascular balloon occlusion of the aorta through the femoral artery, application of Abdominal Aortic Junctional Tourniquet. or other similar instruments like pelvic binder which induces local pressure on the abdominal wall, and injection of intra-abdominal self-expanding foam.

Each of the current interventions has its own drawbacks. The endovascular balloon insertion, which has gained popularity in recent years, entails advanced skills and most of the time is performed under radiologic imaging like ultrasound and completion x-ray imaging. Therefore, endovascular balloon insertion is not suitable for pre-hospital resuscitation. Moreover, the intra-arterial balloon can harm the arterial wall by dissection, as indicated by the many cases of leg ischemia, which led to limb loss due to vessel injury, as a result of this treatment.

The other devices mentioned above, like the pelvic binder and the Abdominal Aortic Junctional Tourniquet, inflict non-focused pressure on the abdominal and pelvic walls and are used mainly to control pelvic or groin hemorrhage. However, such devices are not efficient in controlling intra-abdominal bleeding. In addition, these devices have to be removed before a definitive surgical procedure is done, causing increase in internal bleeding until blood vessel control is gained. This period of increased bleeding subjects the patient to fatal outcome.

### SUMMARY

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present subject matter, suitable methods and materials are described below. In case of conflict, the patent specification, including definitions, will control. In addition, the materials, methods, and examples are illustrative only and not intended to be limiting.

According to one aspect of the present subject matter, there is provided a system for temporarily stopping blood flow through a blood vessel, the system comprising:
at least one magnetically attractive element configured to be introduced into an internal part of a body of a patient, and temporarily placed near a first blood vessel that supplies blood to at least one second blood vessel; and
at least one magnetic field generator configured to be placed outside a body of the patient, and generate a magnetic field that attracts the at least one magnetically attractive element in a manner that the at least one magnetically attractive element presses the first blood vessel, thereby stopping flow of blood through the first blood vessel toward the second blood vessel.

According to one embodiment, the system further comprising a magnetic shield that is configured to be placed in a vicinity of the magnetic field generator, and at least partially reduce a strength of the magnetic field generated by the magnetic field generator, at least in the direction of the body of the patient.

According to yet another embodiment, the system further comprising at least one carrier configured to be inserted into the internal part of the body of the patient, the carrier carrying the at least one magnetically attractive element.

According to still another embodiment, the carrier is elastic.

According to a further embodiment, the carrier is configured to be inserted into a gastrointestinal system of the patient.

According to yet a further embodiment, a gastric inflatable balloon is attached to a distal edge of the carrier, wherein the gastric inflatable balloon is configured to be inserted into a stomach of the patient.

According to still a further embodiment, the at least one magnetically attractive element is attached to the carrier, just above the gastric inflatable balloon.

According to an additional embodiment, the carrier further comprises an esophageal inflatable balloon above the gastric inflatable balloon.

According to yet an additional embodiment, the at least one magnetically attractive element is placed in the esophageal inflatable balloon.

According to still an additional embodiment, the gastric inflatable balloon is configured to be inserted into the stomach of the patient, inflated in the stomach, and fix the carrier in a manner that the esophageal inflatable balloon is placed in the esophagus near an entry to the stomach, and the at least one magnetically attractive element that is in the esophageal inflatable balloon is configured to press an aorta substantially at a border line between the thoracic aorta and the abdominal aorta, thereby stopping flow of blood to blood vessels lower that the border line between the thoracic aorta and the abdominal aorta.

According to another embodiment, the system further comprising a garment configured to be attached to the magnetic field generator, and be worn by the patient in a manner that places the magnetic field generator in a desired location near the body of the patient.

According to another aspect of the present subject matter, there is provided a kit for temporarily stopping blood flow through a blood vessel, the kit comprising the system as described above.

According to still another aspect of the present subject matter, there is provided a method for temporarily stopping blood flow through a blood vessel, the method comprising:
introducing at least one magnetically attractive element into an internal part of a body of a patient;
temporarily placing the at least one magnetically attractive element near a first blood vessel that supplies blood to at least one second blood vessel;
placing at least one magnetic field generator in a vicinity and externally to a body of the patient; and
attracting the at least one magnetically attractive element with the magnetic field generator in a manner that the magnetically attractive element presses the first blood vessel, thereby stopping flow of blood through the first blood vessel toward the second blood vessel.

According to one embodiment, the method further comprising, before the attracting the at least one magnetically attractive element, activating the magnetic field generator.

According to another embodiment, the method further comprising, after the attracting the at least one magnetically attractive element with the magnetic field generator, periodically stopping attraction of the at least one magnetically attractive element by the at least one magnetic field generator.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms may be embodied in practice.

In the drawings:

FIG. 1, schematically illustrates, according to an exemplary embodiment, a system for temporarily stopping blood flow through a blood vessel.

FIG. 2 schematically illustrates, according to an exemplary embodiment, a magnetically attractive element comprising a container filled with a powder of magnetically attractive particles, or a suspension of the powder of magnetically attractive particles.

FIGS. 3A-D, schematically illustrate a block diagram of some exemplary embodiments of a control system for controlling operation of an electromagnet.

FIG. 4 schematically illustrates, according to an exemplary embodiment, a carrier.

FIG. 5 schematically illustrates, according to an exemplary embodiment, a carrier comprising a gastric inflatable balloon inserted into an esophagus and stomach of a patient.

FIG. 6, schematically illustrating, according to an exemplary embodiment, a carrier in a form of a Sengstaken-Blackmore tube comprising at least one magnetically attractive element.

FIG. 7 schematically illustrates, according to an exemplary embodiment, a carrier in a form of a Sengstaken-Blackmore tube inserted into an esophagus and stomach of a patient.

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale.

For clarity, non-essential elements were omitted from some of the drawings.

The present subject matter provides a system, and a method, for temporarily stopping blood flow through a blood vessel, for example in order to temporarily stop internal bleeding, preferably intra-abdominal bleeding, or any other un-controlled internal bleeding. The system and method of the present subject matter are suitable also for emergency treatment of any type of shock, for example shock resulting from loss of blood due to bleeding, septic shock, some kinds of cardiogenic shock, and the like.

The system and method of the present subject matter are suitable for usage in medical care facilities, like hospitals, emergency rooms, medical clinics, and the like. However, an advantage of the system and method of the present subject matter is that they are preferably suitable for usage out of the medical care facilities, for example in battlefields for treating injured soldiers suffering from internal bleeding, traffic accident sites for treating injured individuals suffering from internal bleeding, patient's home for treating patients suffering from rupture of aneurysm or post-partum bleeding, and the like, where it is impossible to provide a definitive medical treatment like in a medical care facility. Another advantage of the system and method of the present subject matter is that the temporary stopping of the blood flow through a blood vessel can be performed by ordinary medical staff without special surgical skills and without the need for imaging, which are requirements of currently available prior art procedures of stopping blood flow through a blood vessel, for example in order to stop internal bleeding.

Since the stopping of blood flow through a blood vessel by the system and method of the present subject matter is temporary, the system and method of the present subject matter can be used as an emergency medical system and procedure for temporarily stopping blood flow through a blood vessel, for example in order to stop internal bleeding in a patient, until the patient is brought to a medical care facility where a definitive treatment can be provided for permanent stopping of the internal bleeding and repairing of injured blood vessels.

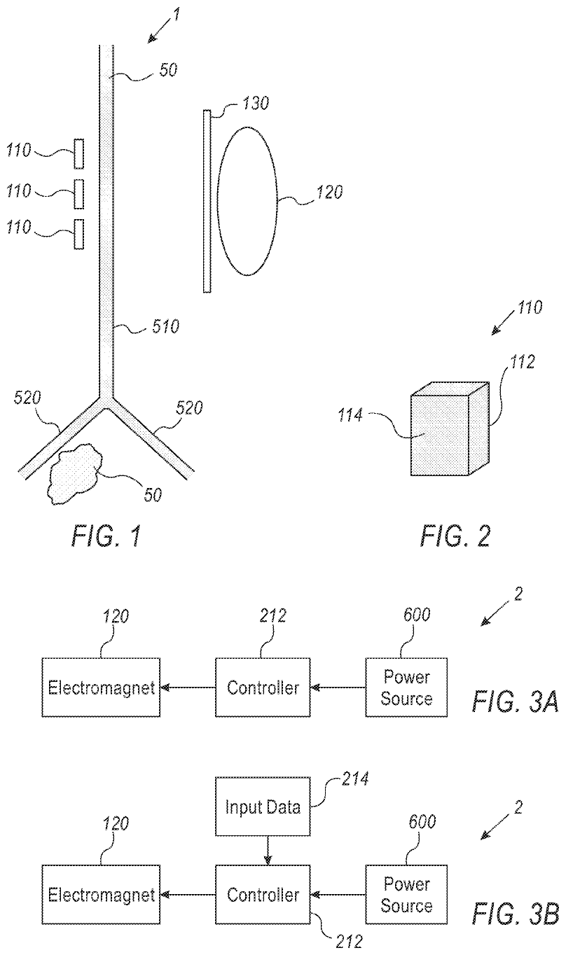

Referring now to FIG. 1, schematically illustrating, according to an exemplary embodiment, a system for temporarily stopping blood flow through a blood vessel. FIG. 1 shows a first blood vessel 510 and a second blood vessel 520, when the direction of flow of blood 50 is from the first blood vessel 510 to the second blood vessel 520, as indicated with arrows 910. In some occasions, the second blood vessel 520 can be injured, resulting in spill of blood 50 out of the injured second blood vessel 520. In addition. FIG. 1 shows components of the system 1 for temporarily stopping blood flow through a blood vessel, including at least one magnetically attractive element 110, and a magnetic field generator 120.

The system 1 for temporarily stopping blood flow through a blood vessel comprises:

at least one magnetically attractive element 110 configured to be introduced into an internal part of a body of a patient, and temporarily placed near a first blood vessel 510 that supplies blood 50 to at least one second blood vessel 520; and

at least one magnetic field generator 120 configured to be placed outside a body of the patient, and generate a magnetic field that attracts the at least one magnetically attractive element 110 in a manner that the at least one magnetically attractive element 110 presses the first blood vessel 510, thereby stopping flow of blood 50 through the first blood vessel 510 toward the second blood vessel 520.

The method for temporarily stopping blood flow through a blood vessel comprises:

introducing at least one magnetically attractive element 110 into an internal part of a body of a patient;

temporarily placing the at least one magnetically attractive element 110 near a first blood vessel 510 that supplies blood 50 to at least one second blood vessel 520;

placing at least one magnetic field generator 120 in a vicinity and externally to a body of the patient; and

attracting the at least one magnetically attractive element 110 with the magnetic field generator 120 in a manner that the magnetically attractive element 110 presses the first blood vessel 510, thereby stopping flow of blood 50 through the first blood vessel 510 toward the second blood vessel 520.

Stopping of blood 50 flow through the first blood vessel 510 can be used for example to stop bleeding from at least one injured second blood vessel 520, or from a site in the first blood vessel 510 that is fed with blood 50 from the site of pressing with the at least one magnetically attractive element 110.

When there is a desire to renew blood 50 flow through the first blood vessel 510, for example when the patient is given definitive treatment for internal bleeding, for example a surgical procedure, and the at least one injured second blood vessel 520 is repaired, or the cause of internal bleeding is treated in another way, there is a desire to terminate the temporary stopping of blood flow through the first blood vessel 510 by the system 1. This is achieved by stopping the attraction of the at least one magnetically attractive element 110 with the magnetic field generator 120, according to embodiments that will be described hereinafter. As a result, the pressing of the first blood vessel 510 by the at least one magnetically attractive element 110 is released, and blood is allowed to flow through the first blood vessel 510.

According to one embodiment, the patient is an animal, for example a vertebrate. According to another embodiment, the patient is a human.

The magnetically attractive element 110 is made of any material that is configured to be attracted by a magnet. According to one embodiment, the magnetically attractive element 110 is made of a ferromagnetic material, for example iron, nickel, cobalt, and the like. According to another embodiment, the magnetically attractive element 110 is a magnet.

According to one embodiment, the magnetically attractive element 110 is a solid piece of material that is configured to be attracted by a magnet. The magnetically attractive element 110 can have any structure that renders it suitable for pressing the first blood vessel 510 and thereby stopping flow of blood 50 toward the second blood vessel 520. Some exemplary structures of the magnetically attractive element 110 are a sphere, a disc and the like. It should be mentioned that the system 1 for stopping blood flow through a blood vessel can comprise one magnetically attractive element 110, or a plurality of magnetically attractive elements 110, or at least one magnetically attractive element, as described above.

According to one embodiment, the magnetically attractive element 110 is in a form of a powder of magnetically attractive particles, for example, microparticles or nanoparticle, made of a material that is configured to be attracted by a magnet. According to another embodiment, the magnetically attractive element 110 is a suspension of the aforementioned powder of magnetically attractive particles in a liquid. According to this embodiment, the system 1 for temporarily stopping flow of blood though a blood vessel further comprises a container configured to contain the powder of magnetically attractive particles, or a suspension of the powder of magnetically attractive particles.

Referring now to FIG. 2, schematically illustrating, according to an exemplary embodiment, a magnetically attractive element comprising a container filled with a powder of magnetically attractive particles, or a suspension of the powder of magnetically attractive particles. FIG. 2 shows the magnetically attractive element 110 comprising a container 112 filled with a powder 114 of magnetically attractive particles, or a suspension of the powder 114 of magnetically attractive particles.

According to one embodiment, the size of magnetically attractive element 110, either the size of the solid piece of material, or the size of the container 112, renders it suitable for pressing the first blood vessel 510 and thereby stopping flow of blood 50 to the second blood vessel 520. An exemplary size of a disc-shaped magnetically attractive element 110 is a diameter of substantially 10 mm and a width of substantially 5 mm. Nevertheless, it should be noted that this exemplary size should not be considered as limiting the scope of the present subject matter.

According to one embodiment, the internal part of the body is the abdominal cavity. According to another embodiment, the internal part of the body is an internal organ. According to yet another embodiment, the internal part of the body is the gastrointestinal system. According to a further embodiment, the internal part of the body is an organ of the gastrointestinal system, for example esophagus, stomach, recto-sigmoid colon, and the like.

Regarding the temporary placement of the at least one magnetically attractive element 110 near the first blood vessel 510, the site of placement supplies blood 50 to the second blood vessel 520. Thus, according to one embodiment, the first blood vessel 510 that supplies blood 50 to the at least one second blood vessel 520 is an artery. According to another embodiment, the first blood vessel 510 is the aorta. On the other hand, the second blood vessel 520 can be any type of blood vessel—the aorta, an artery, a vein, and the vena cava. Stopping supply of blood 50 through an artery, or the aorta, stops flow of blood 50 through arteries, and veins as well, that receive blood 50 from the artery or aorta.

An advantage of the present subject matter is that there is no need to identify the exact site of internal bleeding in order to successfully stop the internal bleeding with the system 1 and method of the present subject matter. This is because the supply of blood 50 to the site of bleeding is stopped by the system 1 and method of the present subject matter. Thus, according to some embodiments, all a user of the system 1 has to do is to place the at least one magnetically attractive element 110 near the aorta, in a place from which blood 50 flows toward the site of bleeding.

According to one embodiment, the magnetic field generator 120 is a magnet. According to another embodiment, the magnetic field generator 120 is an electromagnet 120. According to yet another embodiment, the magnetic field generator 120 is configured to generate a magnetic field that causes the at least one magnetically attractive element 110 to press the first blood vessel 510 in a pressure that causes stopping of flow of blood 50 through the first blood vessel 510. According to a further embodiment, the pressure is up to substantially 1 kg/cm². According to yet a further embodiment, the pressure is in a range of substantially 0.5-1 kg/cm². It should be noted, though, that these values of pressure are only exemplary, and should not be considered as limiting the scope of the present subject matter.

Returning now to FIG. 1. According to one embodiment, the system 1 for temporarily stopping blood flow through a blood vessel further comprises a magnetic shield 130 that is configured to be placed in a vicinity of the magnetic field generator 120 and at least partially reduce a strength of the magnetic field generated by the magnetic field generator 120, at least in the direction of the body of the patient. The magnetic shield 130 is made of any material that is configured to shield a magnetic field, for example a ferromagnetic material, like iron, nickel, cobalt, and the like.

According to one embodiment, the magnetic shield 130 can be used to reduce strength of the magnetic field generated by the magnetic field generator 120, for example in order to reduce the power of attraction of the at least one magnetically attractive element 110, thereby reducing the level of pressure exerted by the at least one magnetically attractive element 110 of the first blood vessel 510.

Another way of reducing the power of attraction of the at least one magnetically attractive element 110 is by increasing the distance between the at least one magnetically attractive element 110 and the magnetic field generator 120. This can be achieved, for example, by moving away the magnetic field generator 120 from the body of the patient.

Still another way of reducing the power of attraction of the at least one magnetically attractive element 110 is by decreasing the strength of electric current supplied to the magnetic field generator 120 in a form of an electromagnet 120, that is described in detail hereinafter.

Thus, according to one embodiment, the method for temporarily stopping blood flow through a blood vessel further comprises, before the attracting the at least one magnetically attractive element 110, activating the magnetic field generator 120. According to one embodiment, the activating the magnetic field generator 120 is removing the magnetic shield 130 from the vicinity of the magnetic field generator 120, thereby exposing the at least one magnetically attractive element 110 to a magnetic field.

According to another embodiment, when the magnetic field generator 120 is an electromagnet 120, the activating the magnetic field generator 120 is supplying an electric current to the magnetic field generator 120, thereby prompting the magnetic field generator 120 to generate a magnetic field. It should be noted that the embodiment of the system 1 further comprising a magnetic shield 130 applies also to the embodiment of the magnetic field generator 120 in the form of an electromagnet 120.

In an embodiment, where the first blood vessel 510 is the aorta, when stopping blood flow through the aorta, for example at a site at the upper abdominal aorta, blood supply is not only stopped to an at least one injured second blood vessel 520, but also to intra-abdominal organs, such as kidneys, liver, spleen (e.g., splenic bleeding after trauma is very common), small intestine, large intestine, and the like. When blood supply to the intra-abdominal organs is stopped for a certain period of time, it can cause ischemic injuries to the intra-abdominal organs, and eventually death of the intra-abdominal organs. For example, stopping blood supply to the intestines for more than substantially 60 minutes can lead to death of the intestines. In order to prevent such ischemic injuries to the intra-abdominal organs, after substantially 60 minutes of stopping blood flow through the aorta, the magnetic field generator 120 can be removed from the vicinity of the patient's body in order to release the pressure on the aorta by the at least one magnetically attractive element 110, and allow flow of blood to the intra-abdominal organ. This release of blood flow can be performed for a short period of time, for example for a few seconds, just to allow supply of some blood to the intra-abdominal organs, with the price of loosing of a small amount of blood 50 from the at least one injured second blood vessel 520.

Thus, according to one embodiment, the method for temporarily stopping blood flow through a blood vessel further comprises, after the attracting the at least one magnetically attractive element 110 with the magnetic field generator 120, periodically stopping attraction of the at least one magnetically attractive element 110 by the at least one magnetic field generator 120. According to one embodiment, the stopping attraction of the at least one magnetically attractive element 110 is achieved by manually removing the at least one magnetic field generator 120 from the vicinity of the body of the patient. According to another embodiment, the removal of the at least one magnetic field generator 120 can be by a device that is configured to remove the at least one magnetic field generator 120, for example a device that is operated electrically. According to another embodiment, the stopping attraction of the at least one magnetically attractive element 110 is achieved by returning the magnetic shield 130 to the vicinity of the magnetic field generator 120 in a manner that at least partially reduces a strength of the magnetic field generated by the magnetic field generator 120, at least in the direction of the body of the patient. According to yet another embodiment, the stopping attraction of the at least one magnetically attractive element 110 is achieved by manually shutting off the supply of the electric current to the magnetic field generator 120 in the form of an electromagnet 120. According to still another embodiment, the stopping attraction of the at least one magnetically attractive element 110 is achieved automatically, for example by automatic shutting off the supply of the electric current to the magnetic field generator 120 in the form of an electromagnet 120. Embodiments relating to controlling the operation of the electromagnet 120 are described hereinafter.

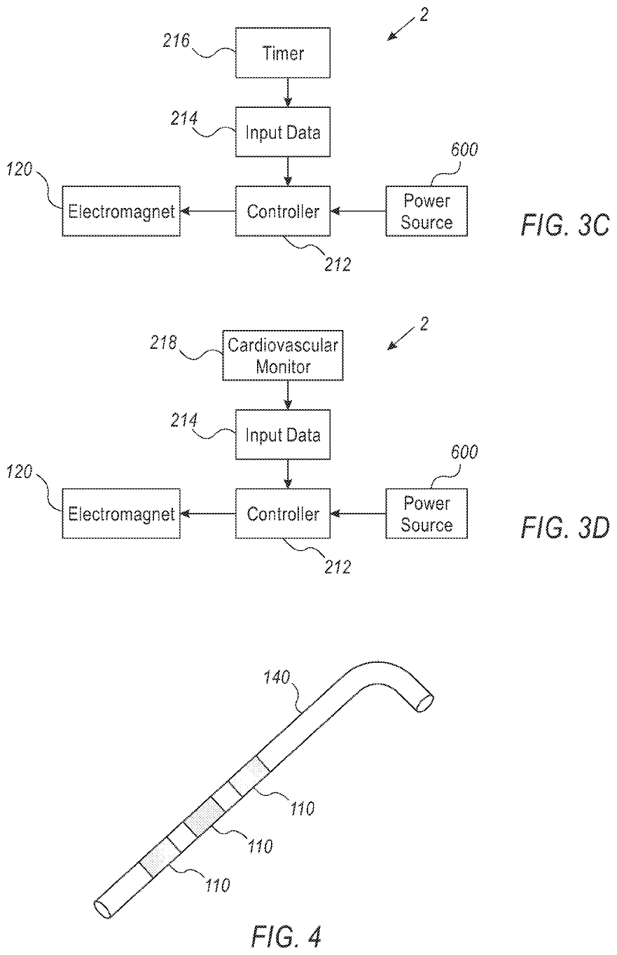

Referring now to FIGS. 3A-D, schematically illustrating a block diagram of some exemplary embodiments of a control system for controlling operation of an electromagnet. FIG. 3A illustrates an exemplary embodiment of a control system 2 for controlling operation of an electromagnet 120, and FIG. 3B illustrates an exemplary embodiment of a control system 2 for controlling operation of an electromagnet 120 according to input data 214.

Referring now to FIG. 3A. According to one embodiment, the system 1 for temporarily stopping internal bleeding further comprises a control system 2 for controlling operation of an electromagnet, the control system 2 comprising:

a power source 600 configured to supply electric current to the electromagnet 120, electrically connected to the electromagnet 120; and

a controller 212 configured to control the supply of electric current from the power source 600 to the electromagnet 120, the controller 212 electrically connected between the power source 600 and the electromagnet 120.

According to one embodiment, the power source 600 is a battery. According to another embodiment, the power source 600 is mains electricity.

According to one embodiment, the electrical connection between the power source 600 and the electromagnet 120, and of the controller 212 in between, is either wired, or wireless.

According to one embodiment, the controller 212 is configured to operate manually by a user. Thus, the user can control the supply of electric current from the power supply 600 to the electromagnet 120 as desired, by manually operating the controller 212.

Referring now to FIG. 3B. According to another embodiment, the controller 212 is configured to operate according to input data 214 received by the controller 212. In other words, according to this embodiment, the controller 212 is configured to operate automatically. According to yet another embodiment, the controller 212 is configured to operate both manually and automatically.

According to one embodiment, the input data 214 is time. In other words, the controller 212 is configured to operate according to input data 214 received from a timer. Referring now to FIG. 3C illustrating a control system 2 further comprising a timer 216 providing time input data 214 to the controller 212. According to one embodiment, the timer 216 is configured to measure length of time, when the length of time is the input data 214 that is received by the controller 212, and the controller 212 is configured to operate according to the time input data 214 received from the timer 216.

According to one embodiment, the input data 214 is a cardiovascular measure, for example heart rate, blood pressure, and the like. In other words, the controller 212 is configured to operate according to input data 214 received from a cardiovascular monitor. Referring now to FIG. 3D illustrating a control system 2 further comprising a cardiovascular monitor 218 providing cardiovascular measure input data 214 to the contoller 212. According to one embodiment, the cardiovascular monitor 218 is configured to measure cardiovascular measures. For example, the cardiovascular monitor 218 can be an electrocardiogram configured to measure heart rate. Another exemplary cardiovascular monitor 218 is a blood pressure meter configured to measure blood pressure.

According to one embodiment, the controller 212 is configured to switch on and off supply of electric current from the power source 600 to the electromagnet 120, either manually, by a user, as shown in FIG. 3A, or according to input data 214 as shown in FIG. 3B. An exemplary input data 214 is time input data 214, received for example from a timer 216, as shown in FIG. 3C.

Here is an example of switching on and off supply of electric current from the power source 600 to the electromagnet 120 according to time input data 214. As mentioned above, in order to prevent ischemic injuries to the intra-abdominal organs, pressure on the aorta by the at least one magnetically attractive element 110 is released for a short period of time, after a certain time of pressing the aorta with the at least one magnetically attractive element 110. For example, the controller 212 is switched on and allow supply of electric current from the power source 600 to the electromagnet 120 for substantially 60 minutes. After substantially 60 minutes the controller 212 is configured to switch off and stop supply of electric current from the power source 600 to the electromagnet 120. Then, after several seconds, the controller 212 is configured to switch on and renew supply of electric current from the power source 600 to the electromagnet 120.

Here is another example of controlling strength of the magnetic field generated by the electromagnet 120, by controlling the strength of electric current supplied from the power source 600 to the electromagnet 120. Changing the strength of the electric current changes accordingly the strength of the magnetic field generated by the electromagnet 120, for example in order to adjust the pressure that is exerted by the at least one magnetically attractive element 110 on the first blood vessel 510, or in order to fit the strength of the magnetic field to a distance between the electromagnet 120 and the at least one magnetically attractive element 110. Thus, according to one embodiment, the controller 212 is configured to change the strength of electric current that is supplied from the power source 600 to the electromagnet 120. In other words, according to this embodiment, the controller 212 operates like a transformer.

According to one embodiment, illustrated in FIG. 3A, the controller 212 is configured to manually change the strength of the electric current, as desired by a user. For example, the user can change the strength of the electric current according to the distance between the electromagnet 120 and the at least one magnetically attractive element 110, or according to observation of a measures measured by a cardiovascular monitor.

According to another embodiment, the controller 212 is configured to change the strength of the electric current according to input data 214 received by the controller 212, as shown in FIG. 3B. For example, when systolic blood pressure of the patient is low, for example substantially 70 mm Hg, it is a symptom of inter alia an internal bleeding. If during operation of the system 1 for temporarily stopping blood flow through a blood vessel the systolic blood pressure drops to substantially 70 mm Hg, it shows that the at least one magnetically attractive element 110 does not efficiently press the first blood vessel 510. One way to overcome this situation is to increase the strength of the electric current supplied to the electromagnet 120, and thereby increasing the pressure of the at least one magnetically attractive element 110 on the first blood vessel 510. This can be achieved by the control system 2 illustrated in FIG. 3D. The controller 212 receives blood pressure input date 214 from a cardiovascular monitor 218 in a form of a blood pressure meter. When the systolic blood pressure is normal, the controller 212 is configured to let the strength of electric current to remain as is. However, when the systolic blood pressure drops, for example to a level of substantially 70 mm Hg, the controller 212 is configured to increase the strength of the electric current supplied from the power source 600 to the electromagnet 120.

Another example is switching on and off the supply of electric current from the power source 600 to the electromagnet 120 according to heartbeat cycle. The system 1 for temporarily stopping blood flow through a blood vessel can be used also to treat heart failure that is caused by poor blood supply to the coronary arteries. In this example, the at least one magnetically attractive element 110 is placed in the vicinity of the aorta, substantially near the border line between the thoracic aorta and the abdominal aorta. In order to increase blood supply to the coronary arteries there is a desire to block passage of blood in the border line between the thoracic aorta and the abdominal aorta during diastole. As a result, more blood is directed toward the coronary arteries. Then, during systole, there is a desire to release blood flow through the aorta in order to supply blood to the abdominal arteries and the other lower arteries. This is achieved by switching on and off the supply of electric current from the power source 600 to the electromagnet 120 according to the heartbeat cycle. In this example, the controller 212 is configured to receive pulses of blood pressure during heartbeat cycle, for example from a cardiovascular monitor 218, as shown in FIG. 3D, when the cardiovascular monitor 218 is in a form of an electrocardiogram. Thus, during systole, the controller 212 is configured to switch off supply of electric current to the electromagnet 120, resulting in no pressure exerted on the aorta; while during diastole, the controller 212 is configured to switch on supply of electric current to the electromagnet 120, resulting in pressure exerted by the at least one magnetically attractive element 110 on the aorta, substantially in the border line between the thoracic aorta and the abdominal aorta, and as a result increase blood supply to the coronary arteries during diastole.

According to some embodiments, the treatment of heart failure described above can also be performed by controlling the strength of the magnetic field to which the at least one magnetically attractive element 110 is exposed also when the magnetic field generator 120 is a magnet 120. For example, the system 1 for temporarily stopping blood flow through a blood vessel can further comprises a mechanism that controls the distance between the magnet 120 and the at least one magnetically attractive element 110, or practically a mechanism that controls the distance between the magnet 120 and the body of the patient. Thus, during systole the magnet 120 is moved away from the body of the patient in order to decrease the strength of, or abolish, the magnetic field to which the at least one magnetically attractive element 110 is exposed, thereby decreasing, or abolishing, the pressure exerted on the aorta. On the other hand, during diastole the magnet 120 is returned to its position in the vicinity of the patient's body in order to increase the strength of the magnetic field to which the at least one magnetically attractive element 110 is exposed, thereby increasing the pressure exerted on the aorta.

Another embodiment for treating heart failure, when the magnetic field generator 120 is a magnet 120, is by using a mechanism that controls position of the magnetic shield 130. Thus, during systole, the magnetic shield 130 is placed between the magnet 120 and the patient's body in order to decrease the strength of, or abolish, the magnetic field to which the at least one magnetically attractive element 110 is exposed, thereby decreasing, or abolishing, the pressure exerted on the aorta. On the other hand, during diastole the magnetic shield 130 is removed from its place between the magnet 120 and the patient's body in order to increase the strength of the magnetic field to which the at least one magnetically attractive element 110 is exposed, thereby increasing the pressure exerted on the aorta.

Referring now to FIG. 4, schematically illustrating, according to an exemplary embodiment, a carrier. FIG. 4 illustrates a carrier 140 configured to carry at least one magnetically attractive element 110.

According to one embodiment, the system 1 for temporarily stopping blood flow through a blood vessel further comprises at least one carrier 140 configured to be inserted into an internal part of a body of a patient, the carrier 140 carrying at least one magnetically attractive element 110. According to another embodiment, the carrier 140 has an elongated structure. According to yet another embodiment, the carrier 140 is elastic in a manner that allows passage of the carrier 140 through cavities and corners in the body of the patient.

According to one embodiment, the at least one magnetically attractive element 110 is a part of the carrier 140. In other words, most of the carrier 140 is made of a material that is not attracted by a magnet. However at least one part of the carrier 140 is made of a material that is configured to be attracted by a magnet, thus defining at least one magnetically attractive element 110.

According to another embodiment, the at least one magnetically attractive element 110 is made according to embodiments described above, and attached to the carrier 140 that is made of a material that is not attracted by a magnet. The attachment of the at least one magnetically attractive element 110 to the carrier 140 is achieved by any mechanism or method, for example adhering, gluing, welding, and the like.

According to one embodiment, the carrier 140 has a form of a tube 140. According to another embodiment, the at least one magnetically attractive element 110 resides inside a lumen of the tube 140. According to yet another embodiment, the at least one magnetically attractive element 110 is attached to the tube 140, as described above.

According to one embodiment, the carrier 140 is configured to be inserted into a gastrointestinal system of the patient. According to a preferred embodiment, the carrier 140 that is configured to be inserted into the gastrointestinal system of the patient is in a form of a tube 140. According to another embodiment, the tube 140 is configured to be inserted into the esophagus and stomach of the patient.

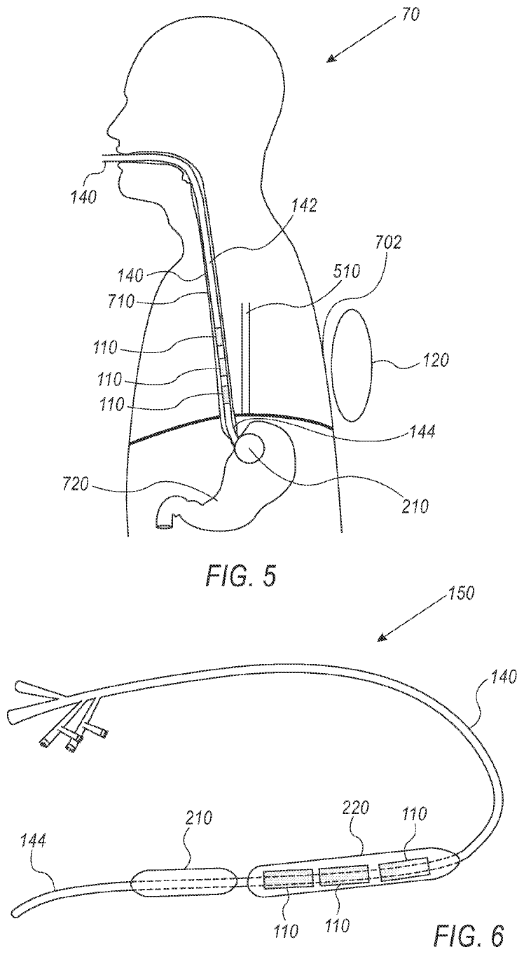

Referring now to FIG. 5, schematically illustrating, according to an exemplary embodiment, a carrier comprising a gastric inflatable balloon inserted into an esophagus and stomach of a patient. FIG. 5 illustrates embodiments of a carrier 140 that is configured to be inserted into the esophagus 710 and stomach 720 of a patient 70. Even though the carrier 140 illustrated in FIG. 4 can be used for insertion into the esophagus 710 and stomach 710, the tube 140 illustrated in FIG. 5 is more suitable for this purpose.

Insertion of the at least one magnetically attractive element 110 into the esophagus 710 and stomach 720 with the carrier 140 comprising a gastric inflatable balloon 210 is advantageous, because the aorta 510, which is considered now as a first blood vessel 510 as disclosed herein, is close to the esophagus 710, particularly the border line between the thoracic aorta and the abdominal aorta is adjacent to the distal edge of the esophagus 710, just before entering into the stomach 720. As a result, it is advantageous to place the at least one magnetically attractive element 110 in the distal edge of the esophagus 710. A procedure of placing the at least one magnetically attractive element 110 in the distal edge of the esophagus 710 with the carrier 140 illustrated in FIG. 5, is advantageous because it is considered non-invasive, and easy to perform, with no need of high surgical professional experience and equipment, compared, for example, to a procedure involving puncturing of the thorax or abdomen, inserting through the created hole the at least magnetically attractive element 110 into the thoracic or abdominal cavity, and placing the at least one magnetically attractive element 110 in a vicinity of a first blood vessel 510, for example with the carrier 140 illustrated in FIG. 4.

Referring now to FIG. 5. According to one embodiment, the carrier 140 is a tube 140 having a lumen 142, and a distal edge 144. The carrier 140 is configured to be inserted into the esophagus 710 and stomach 720 of a patient 70, when the distal edge 144 of the carrier 140 is configured to be inserted into the stomach 720. A gastric inflatable balloon 210 is attached to the distal edge 144 of the carrier 140 an is also configured to be inserted into the stomach 720. Just above the gastric inflatable balloon 210, at least one magnetically attractive element 110 is attached to the carrier 140, for example, according to one embodiment by being inserted into the lumen 142 of the carrier 140, and according to another embodiment, attached to a wall of the carrier 140. The attachment of the at least one magnetically attractive element 110 to the carrier 140 is important in order to hold the at least one magnetically attractive element 110 in its proper preferable place-just above the gastric inflatable balloon 210.

It should be noted though that the preferred position of the at least one magnetically attractive element 110 just above the gastric inflatable balloon 210 is only exemplary, and should not be considered as limiting the scope of the present subject matter. The at least one magnetically attractive element 110 can be placed anywhere along the carrier 140, as well as inside the gastric inflatable balloon 210.

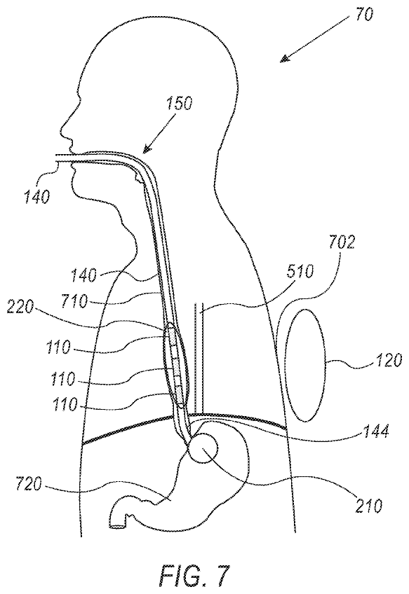

Referring now to FIG. 6, schematically illustrating, according to an exemplary embodiment, a carrier in a form of a Sengstaken-Blackmore tube comprising at least one magnetically attractive element, and to FIG. 7 schematically illustrating, according to an exemplary embodiment, a carrier in a form of a Sengstaken-Blackmore tube inserted into an esophagus and stomach of a patient.

According to one embodiment, the Sengstaken-Blackmore tube 150, that is configured to be inserted into the esophagus 710 and stomach 720 can be adapted for use as a carrier 140 according to embodiments of the present subject matter. The Sengstaken-Blackmore tube 150 comprises a tube 140; a gastric inflatable balloon 210, similar to the gastric inflatable balloon 210 shown in FIG. 5, that is configured to be inserted into the stomach 720; and an esophageal inflatable balloon 220, configured to be inserted and remain in the esophagus 710, near the entry of the esophagus 710 into the stomach 720. The position of the esophageal inflatable balloon 220 in the Sengstaken-Blackmore tube 150 is similar to the position of the at least one magnetically attractive element 110 in the carrier 140 comprising a gastric inflatable balloon 210, shown in FIG. 5. Therefore, the Sengstaken-Blackmore tube 150 can be adapted for use a carrier 140, in its preferred embodiment according to present subject matter, by placing the at least one magnetically attractive element 110 in the esophageal inflatable balloon 220, as illustrated in FIG. 6. Thus, according to one embodiment, the carrier 140 that comprises a gastric inflatable balloon 210, further comprises an esophageal inflatable balloon 220 above the gastric inflatable balloon 210.

As can be seen in FIG. 7, the Sengstaken-Blackmore tube 150 further comprising at least one magnetically attractive element 110 is inserted into the esophagus 710 and stomach 720 of the patient 70. The gastric inflatable balloon 210 is inserted into the stomach 720, similarly to the gastric inflatable balloon 210 shown in FIG. 5. As a result, the esophageal inflatable balloon 220, that comprises the at least one magnetically attractive element 110, is placed in the esophagus 710 near the entry into the stomach 720. Thus, the at least one magnetically attractive element 110 is placed in the preferred location shown in FIG. 5, aside the border line between the thoracic aorta and the abdominal aorta.

It should be noted though that the preferred position of the at least one magnetically attractive element 110 in the esophageal inflatable balloon 220 is only exemplary, and should not be considered as limiting the scope of the present subject matter. The at least one magnetically attractive element 110 can be placed anywhere along the tube 140 of the Sengstaken-Blackmore tube 150, as well as inside the gastric inflatable balloon 210.

The present subject matter provides a method for temporarily stopping blood flow through a blood vessel substantially at a border line between a thoracic aorta and an abdominal aorta. This method is based on the aforementioned method for temporarily stopping blood flow through a blood vessel, with the following embodiments.

According to one embodiment, the at least one magnetically attractive element 110 is carried by a carrier 140 comprising a gastric inflatable balloon 210, according to embodiments described above.

According to one embodiment, the internal part of a body is an esophagus and a stomach.

According to one embodiment, the first blood vessel 510 is substantially a border line between a thoracic aorta and an abdominal aorta.

According to one embodiment, the temporarily placing the at least one magnetically attractive element 110 near a first blood vessel 510, namely substantially at a border line between a thoracic aorta and an abdominal aorta, comprising:

inserting the carrier 140 comprising a gastric inflatable balloon 210 into an esophagus 710 of a patient 70, wherein the inflatable balloon 210 is deflated, until the gastric inflatable balloon 210 is inserted into a stomach 720 of the patient 70:

inflating the gastric inflatable balloon 210 to a size that is larger than the entry site of the esophagus 710 to the stomach 720;

pulling the carrier 140 outside until the inflated gastric inflatable balloon 210 is blocked by the entry site of the esophagus 710 to the stomach 720.

According to one embodiment, the inflating the gastric inflatable balloon 210 is with a gas. According to another embodiment, the gas is air.

According to one embodiment, the gastric inflatable balloon 210 is inflated to a volume of substantially 50 ml, or substantially 200 ml, or in a range of substantially 50-200 ml.

According to one embodiment, when the esophageal inflatable balloon 220 is placed in its desired position, the esophageal inflatable balloon 220 remains deflated. In other words, the esophageal inflatable balloon 220 is not inflated. According to another embodiment, the esophageal inflatable balloon 220 is inflated with a gas, for example air: or liquid, for example a suspension of magnetically attractive particles. According to yet another embodiment, the esophageal inflatable balloon 220 is inflated up to a pressure of substantially 30 mm Hg.

According to one embodiment, after the pulling the carrier 140 outside—fixing the carrier 140, for example by tying the carrier 140 around a head of the patient 70 with a thread, or fixing the carrier 140 to a face or neck of the patient 70 with an adhesive bandage, and the like.

As can be seen in FIG. 5, inserting the carrier comprising a gastric inflatable balloon 210 as described in the aforementioned method ensures that the at least one magnetically attractive element 110 is placed in the esophagus 710, close to the entry into the stomach 720. Thus, the at least one magnetically attractive element 110 is placed at its desired location—in close vicinity substantially at a border line between a thoracic aorta and an abdominal aorta. As described in detail hereinafter, the location of the at least one magnetically attractive element 110 can be confirmed by using a metal detector that is placed externally to the body of the patient.

The next step of the method is placing at least one magnetic field generator 120 in a vicinity and externally to a body of the patient 70. As illustrated in FIG. 5, the at least one magnetic field generator 120 is placed, for example, near a back 702 of the patient 70, at the level of the at least one magnetically attractive element 110. More particularly, the at least one magnetic field generator 120 can be placed externally, on a back midline, opposite to a first lumbar vertebra, also known as LI vertebra. Thus, the at least one magnetically attractive element 110 is attracted toward the at least one magnetic field generator 120, in the direction of the aorta 510, thereby pressing the aorta 510 and stopping flow of blood through the aorta 510. Furthermore, at this site the aorta 510 passes aside the spine that resides between the aorta 510 and the at least one magnetic field generator 120. Therefore, the at least one magnetically attractive element 110 presses the aorta 510 against at least one vertebra of the spine, thus increasing the effect of pressure on the aorta 510 and the efficiency of stopping the flow of blood 50.

Another example relates to pressing an abdominal aorta. According to this example, the at least one magnetically attractive element 110 is placed inside the gastric inflatable balloon 210 of the carrier 140 described in FIG. 5, or in the gastric inflatable balloon 210 of the Sengstaken-Blackmore tube 150 comprising at least one magnetically attractive element 110, illustrated in FIGS. 6 and 7. During the method for temporarily stopping blood flow through a blood vessel, the carrier 140, or the Sengstaken-Blackmore tube 150, is inserted into the esophagus 710 and stomach 720 as described above, except that the gastric inflatable balloon 210 is inserted into a lower position in the stomach 720, in a manner that also the at least one magnetically attractive element 110 is inserted into the stomach 720. In the case of the Sengstaken-Blackmore tube 150, the esophageal inflatable balloon 220, in which the at least one magnetically attractive element 110 resides, is not inflated, and is also inserted into the stomach 720. Then, the at least one magnetic field generator 120 is placed at a lower level on the back midline, opposite the spine. As a result, the at least one magnetically attractive element 110 presses a lower part of the abdominal aorta.

According to one embodiment, the carrier 140 is Sengstaken-Blackmore tube 150 comprising at least one magnetically attractive element 110. According to a preferred embodiment, the at least one magnetically attractive element 110 is placed inside the esophageal inflatable balloon 220 of the Sengstaken-Blackmore tube 150.

According to one embodiment, the carrier 140 is inserted into the esophagus 710 and stomach 720 through a mouth of the patient 70. According to another embodiment, the carrier 140 is inserted into the esophagus 710 and stomach 720 of through a nose of the patient 70.

According to one embodiment, in the method for temporarily stopping blood flow through a blood vessel, the internal part of the body into which the at least one magnetically attractive element 110 is introduced, is a sigmoid colon. Accordingly, also the carrier 140, and the carrier 140 in the form of the Sengstaken-Blackmore tube 150, are configured to be inserted into the sigmoid colon, for example through the anus. This embodiment allows temporary stopping of bleeding in a lower area of the body of the patient, in the pelvis, groins, or upper legs. In order to stop blood flow in this area, the magnetic field generator 130 is positioned behind the spine of the patient, outside and near the patient's body, at a level similar to the level of the at least one magnetically attractive element 110 that is inside the sigmoid colon. Attraction of the at least one magnetically attractive element 110 by the magnetic field generator 130 cause the at least one magnetically attractive element 110 to press a blood vessel that resides adjacent to the sigmoid colon, and stop blood flow through this blood vessel.

Another example of using the at least one magnetically attractive element 110 that is placed in the sigmoid colon is as follows: In this state, the at least one magnetically attractive element 110 is compressed against the sigmoid colon and a lower lumbar vertebra of the spine. In order to bring the at least one magnetically attractive element 110 to the blood vessel that has to be pressed, a magnetic field generator 120 is used to mobilize the sigmoid colon with the at least one magnetically attractive element 110 to a desired location, for example under the umbilicus from outer the abdominal wall anteriorly. Then, another magnetic field generator 120 is placed on the midline lower back of the patient, on the level of the umbilicus. As a result, the aortic bifurcation is pressed and blood flow through it is stopped.

Returning now to FIGS. 4-7. As illustrated in these drawings, when multiple magnetically attractive elements 110 are used, there is a gap between two adjacent magnetically attractive elements 110. When the carrier is elastic, these gaps allow flexibility of the carrier 140 also where the magnetically attractive elements 110 are placed. This is important because in some embodiments, the carrier 140 is inserted into internal cavities of the body, and has to be elastic in order to be able to pass through corners of the cavities. For example, as illustrated in FIGS. 5 and 7, when the carrier 140, or the Sengstaken-Blackmore tube 150, respectively, is inserted into the esophagus 710, it has to bend when passing from the mouth or nose into the esophagus 710. The gaps between the magnetically attractive elements 110 allow this bending, for example when the magnetically attractive elements 110 are solid. Thus, according to one embodiment, there is a gap between two adjacent magnetically attractive elements 110. According to another embodiment, the gap is filled with an elastic material, for example silicon, or a spacer made of an elastic material is placed between two adjacent magnetically attractive elements 110.

Returning now to FIGS. 5 and 7. As described above, the gastric inflatable balloon 210 can be used to place the at least one magnetically attractive element 110 in a desired location, when for example the desired location is in the esophagus 710, adjacent to the entry into the stomach 720. However, this method cannot be used when there is a desire to place the at least one magnetically attractive element 110 in other locations in the body. One way to overcome this issue is using a metal detector.

Thus, according to one embodiment, the system 1 for temporarily stopping blood flow through a blood vessel further comprises a metal detector, configured to be placed externally to the body of the patient, and locate a position of the at least one magnetically attractive element 110 inside the body. The metal detector can be used to determine the position of the at least one magnetically attractive element 110 in the body, as well as being used to bring the at least one magnetically attractive element 110 to a desired location in the body.

According to one embodiment, the magnetic field generator 120 is manually placed in a desired place externally to the patient's body, and manually held in place as desired. According to another embodiment, the magnetic field generator 120 can be fixed to its place on the body, for example by using plasters, or any other type of temporary adhesive, or by tying with bands and the like. According to yet another embodiment, the magnetic field generator 120 can is attached to a garment that is configured to be worn by the patient in a manner that places the magnetic field generator 130 in a desired location near the body of the patient. An exemplary garment can be a belt, a vest, and the like. Thus, the system 1 for temporarily stopping blood flow through a blood vessel further comprises a garment configured to be attached to the magnetic field generator 120, and be worn by the patient in a manner that places the magnetic field generator 120 in a desired location near the body of the patient.

The present subject matter further provides a kit for temporarily stopping blood flow through a blood vessel, the kit comprising the components of the system 1 for temporarily stopping blood flow through a blood vessel, according to the embodiments described above.

### Exemplary Case Studies

### Exemplary Case Study 1

A soldier is hit in the abdomen by a bullet that causes an un-controlled spleen hemorrhage that can be fatal if not stopped in a short period of time. In order to stop the hemorrhage, a Sengstaken-Blackmore tube 150 comprising at least one magnetically attractive element 110 is inserted by a paramedic through the mouth into the esophagus and stomach of the wounded soldier, and a magnetic field generator 120 is placed, as illustrated in FIG. 7. The at least one magnetically attractive element 110 that is in the esophageal inflatable balloon 220 compresses the upper abdominal aorta 510 and in this way temporarily stops blood supply to the spleen and the bleeding is stopped. This enables evacuation of the wounded soldier to a field hospital, for example with a helicopter, without any significant blood loss. Up to substantially 60 minutes after the injury of the soldier, he is operated in the field hospital and the bleeding spleen is removed. Without the magnetic aortic compression, the wounded soldier could have died during the evacuation due to exsanguination, before reaching the field hospital.

### Exemplary Case Study 2

A 70 years old male known to have an asymptomatic abdominal aortic aneurysm, collapses and faints in his home. An ambulance was urgently called, and a paramedic from the ambulance feels pulsating big swelling on the abdomen above the umbilicus. Systolic blood pressure is 70 mm Hg. A sudden rupture of abdominal aortic aneurysm is suspected. The paramedic inserts a Sengstaken-Blackmore tube 150 comprising at least one magnetically attractive element 110, as illustrated in FIG. 7, and the esophageal inflatable balloon 220, that contains the at least one magnetically attractive element 110, is inflated in the esophagus. A magnetic field generator 120 is placed near the back 702 of the patient, as shown in FIG. 7, and activated and attracts the at least one magnetically attractive element 110, which compresses and closes the upper abdominal aorta. The bleeding from the ruptured aneurysm is temporarily stopped and the patient is transferred with the ambulance to an urgent life-saving operation in the near hospital, without any loss of blood due to the aortic rupture. Without the magnetic compression, there is a high probability that this patient would have died before reaching the hospital.

### EXPERIMENTAL

Proof of concept of the present subject matter was shown in an animal model. A pig weighing 74 kg was anesthetized and tracheally intubated. A blood pressure transducer was inserted into the femoral artery. A hemodynamic shock was mimicked by reducing the systolic blood pressure of the pig to substantially 70 mm Hg with anesthetizing gases. Then, a Sengstaken-Blackmore tube 150 comprising at least one magnetically attractive element 110, as illustrated in FIG. 6 was inserted into the esophagus and stomach of the pig, as shown in FIG. 7. The gastric inflatable balloon 210, that was inserted into the stomach, was inflated to a volume of 50 ml, the Sengstaken-Blackmore tube 150 was pulled out till the gastric inflatable balloon 210 was blocked at the entry site of the esophagus into the stomach, and fixed. Then, a magnetic field generator 120 was positioned near the pig's back, adjacent to vertebra T12 of the spine. Once the magnetic field generator 120 was positioned in place, the systolic blood pressure in the femoral artery dropped down to substantially 0 mm Hg, namely the graph of time vs. arterial blood pressure, which is normally sinusoidal, was flattened to a straight line. This drop of blood pressure is an indication that blood flow to the femoral artery was stopped due to the pressure that was exerted by the at least one magnetically attractive element 110, that resided in the esophagus, on the aorta again vertebra T12 of the spine.

It is appreciated that certain features of the subject matter, which are, for clarity, described in the context of separate embodiments, may also be provided in combination in a single embodiment. Conversely, various features of the subject matter, which are, for brevity, described in the context of a single embodiment, may also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A system for temporarily stopping blood flow through a blood vessel, the system comprising:
at least one magnetically attractive element configured to be introduced into an internal part of a body of a patient, and temporarily placed near a first blood vessel that supplies blood to at least one second blood vessel; and
at least one magnetic field generator configured to be placed outside a body of the patient, and generate a magnetic field that attracts the at least one magnetically attractive element in a manner that the at least one magnetically attractive element presses the first blood vessel, thereby stopping flow of blood through the first blood vessel toward the second blood vessel.

2. The system according to claim 1, further comprising a magnetic shield that is configured to be placed in a vicinity of the magnetic field generator, and at least partially reduce a strength of the magnetic field generated by the magnetic field generator, at least in the direction of the body of the patient.

3. The system according to claim 1, further comprising at least one carrier configured to be inserted into the internal part of the body of the patient, the carrier carrying the at least one magnetically attractive element.

4. The system according to claim 3, wherein the carrier is elastic.

5. The system according to claim 3, wherein the carrier is configured to be inserted into a gastrointestinal system of the patient.

6. The system according to claim 3, wherein a gastric inflatable balloon is attached to a distal edge of the carrier, wherein the gastric inflatable balloon is configured to be inserted into a stomach of the patient.

7. The system according to claim 6, wherein the at least one magnetically attractive element is attached to the carrier, just above the gastric inflatable balloon.

8. The system according to claim 6, wherein the carrier further comprises an esophageal inflatable balloon above the gastric inflatable balloon.

9. The system according to claim 8, wherein the at least one magnetically attractive element is placed in the esophageal inflatable balloon.

10. The system according to claim 9, wherein the gastric inflatable balloon is configured to be inserted into the stomach of the patient, inflated in the stomach, and fix the carrier in a manner that the esophageal inflatable balloon is placed in the esophagus near an entry to the stomach, and the at least one magnetically attractive element that is in the esophageal inflatable balloon is configured to press an aorta substantially at a border line between the thoracic aorta and the abdominal aorta, thereby stopping flow of blood to blood vessels lower that the border line between the thoracic aorta and the abdominal aorta.

11. The system of claim 1, further comprising a garment configured to be attached to the magnetic field generator, and be worn by the patient in a manner that places the magnetic field generator in a desired location near the body of the patient.

12. A kit for temporarily stopping blood flow through a blood vessel, the kit comprising the system according to claim 1.

13. A method for temporarily stopping blood flow through a blood vessel, the method comprising:
introducing at least one magnetically attractive element into an internal part of a body of a patient;
temporarily placing the at least one magnetically attractive element near a first blood vessel that supplies blood to at least one second blood vessel;
placing at least one magnetic field generator in a vicinity and externally to a body of the patient; and
attracting the at least one magnetically attractive element with the magnetic field generator in a manner that the magnetically attractive element presses the first blood vessel, thereby stopping flow of blood through the first blood vessel toward the second blood vessel.

14. The method according to claim 13, further comprising, before the attracting the at least one magnetically attractive element, activating the magnetic field generator.

15. The method according to claim 13, further comprising, after the attracting the at least one magnetically attractive element with the magnetic field generator, periodically stopping attraction of the at least one magnetically attractive element by the at least one magnetic field generator.
